(19) United States
(12) Patent Application Publication
(10) Pub. No.: US 2024/0287214 A1
(43) Pub. Date: Aug. 29, 2024

(54) CHLORELLA MANNOGALACTAN OR SULFATED CHLORELLA MANNOGALACTAN COMPOUND AND APPLICATION OF SULFATED CHLORELLA MANNOGALACTAN COMPOUND

(71) Applicant: GUANGXI UNIVERSITY OF CHINESE MEDICINE, Nanning (CN)

(72) Inventors: Longyan ZHAO, Nanning (CN); Qingxia YUAN, Nanning (CN); Hao TANG, Nanning (CN); Jinwen HUANG, Nanning (CN); Kunling LV, Nanning (CN)

(73) Assignee: GUANGXI UNIVERSITY OF CHINESE MEDICINE, Nanning, GX (CN)

(21) Appl. No.: 18/626,334

(22) Filed: Apr. 4, 2024

(30) Foreign Application Priority Data
Feb. 7, 2023 (CN) 2023100722929

(51) Int. Cl. C08B 37/00; A61K 31/737; A61K 36/05

(52) U.S. Cl. CPC C08B 37/0087; A61K 31/737; A61K 36/05

(57) ABSTRACT

Chlorella mannogalactan (CM) or a sulfated Chlorella mannogalactan (SCM) compound and application of the SCM compound are disclosed. The CM is obtained by extracting total polysaccharides from Chlorella dry powder before performing separation and purification. The SCM compound is obtained by sulfation modification of the CM. The CM and the SCM compound both consist of mannose, 3-O-methyl-mannose, and galactose. The SCM compound and an excipient thereof form a pharmaceutical composition that can be used as a lyophilized powder injection or an aqueous solution for injection for prevention and/or treatment of thrombotic cardiovascular diseases. The CM or the SCM compound and the application of the SCM compound are adopted. The SCM has good water solubility and strong anticoagulant activity, which can be comparable to enoxaparin sodium.

### TECHNICAL FIELD

The present disclosure relates to the technical field of medicines, and in particular to Chlorella mannogalactan (CM) or a sulfated Chlorella mannogalactan (SCM) compound and application of the SCM compound.

### BACKGROUND ART

Cardiovascular and cerebrovascular diseases have become the most important diseases endangering the health and lives of residents in China, and have become a major public health problem. Annual Report on Cardiovascular Health and Diseases in China (2021) has shown that with the development of the social economy and changes in the national lives and diets of citizens, the influence of cardiovascular and cerebrovascular diseases on health has become more and more prominent.

A thrombus is one of the important causes of cardiovascular diseases. Blood is an important substance carrier for the metabolism of the human body, so the stability of its flow state is very important. Blood stasis and even thrombus due to various reasons will cause vascular stenosis and occlusion, which will lead to coronary heart disease, cerebral apoplexy, and other serious consequences. Therefore, antithrombosis is an important means of treating the cardiovascular diseases. An anticoagulant drug, a thrombolytic drug, and an antiplatelet drug are the three most demanded antithrombotic drugs at present. The anticoagulant drug can inhibit a blood coagulation (co-) factor, etc., and block a key link in a blood coagulation process to play an antithrombotic effect and an important role in clinical practice. For example, low molecular weight heparin is the most widely used anticoagulant drug in clinical application, with global annual sales of more than 7 billion dollars.

Due to high negative charge density, sulfated polysaccharides exhibit various biological activities, especially strong anticoagulant activity. For example, heparin is a sulfated polysaccharide extracted from intestinal mucosae, livers, and lungs of pigs, sheep, cattle, and other animals. It is the first anticoagulant drug found and applied in clinical practice and has been active in clinical practice for nearly a hundred years. Heparin drugs, such as unfractionated heparin and low molecular weight heparin, widely used in clinical practice are derived from organs of animals such as pigs, sheep, and cattle. The heparin of animal origin is at serious risk of contamination due to the prevalence of mad cow disease, infection with a classical swine fever virus, and other animal epidemic diseases. Many sulfated polysaccharides obtained by artificial modification also have good anticoagulant activity. For example, propylene glycol alginate sodium sulfate (PSS) and propylene glycol mannate sulfate (PGMS) are sulfated derivatives of brown algae polysaccharides, which are mainly used for treating ischemic cerebrovascular disease, coronary heart disease, hyperlipoproteinemia, angina pectoris, and other cardiovascular diseases. However, the sulfated polysaccharides still have some side effects such as conjunctival suffusion, edemas, and fevers, and have common defects of existing anticoagulant drugs in clinical practice: a severe bleeding tendency and other side effects, which seriously affects their clinical application. At present, there is still an urgent need for a safe and effective anticoagulant drug in clinical practice.

Chlorella, including Chlorella pyrenoidosa, Chlorella ellipsoidea, Chlorella emersonii, Chlorella kesslerii, Chlorella vulgaris, etc., has strong environmental adaptability and has become one of the widely cultivated microalgae in China in a large scale with a rich yield. The inventor has found CM from Chlorella of a novel and regular structure and further found that a sulfated derivative (SCM) has a strong anticoagulant activity and is expected to be used for preparing a drug for treating a thrombotic cardiovascular disease. There are no relevant research and reports on the CM of a novel structure, an SCM compound, a preparation method of the SCM compound, and application of the SCM compound in treating the thrombotic cardiovascular disease provided by the present disclosure.

### SUMMARY

An objective of the present disclosure is to provide CM or an SCM compound and application of the SCM compound. The CM is extracted and separated from Chlorella with strong fecundity, rapid growth rate, and high yield to prepare a polysaccharide sulfate derivative, to replace heparin. The CM is not contaminated by viruses, bacteria, etc., and is low in price. The SCM prepared by the CM has strong anticoagulant activity, can effectively inhibit FXase of an intrinsic pathway, and has an important application potential for preventing and treating a thrombotic cardiovascular disease.

To achieve the above objective, the present disclosure provides the CM consisting of mannose, 3-O-methyl-mannose, and galactose, having a general formula as follows:

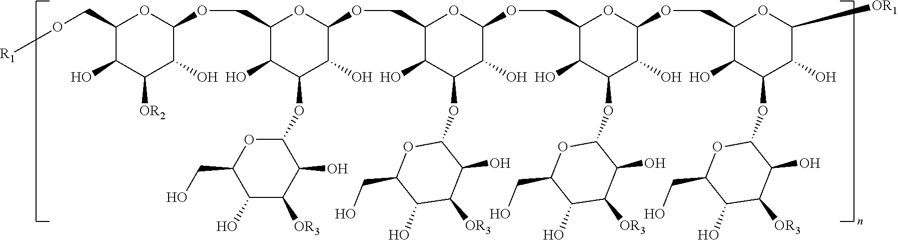

where R₁ is one or more of H or D-β-galactosyl;
R₂ is one or more of the H or D-α-mannosyl or 3-O-methyl-D-α-mannosyl;
R₃ is one or more of the H or —CH₃; and
n is an arbitrary integer of 1-10.

Raw materials are one or more of Chlorella pyrenoidosa dry powder, Chlorella ellipsoidea dry powder, Chlorella emersonii dry powder, Chlorella kesslerii dry powder, and Chlorella vulgaris dry powder; the CM is obtained by extracting total polysaccharides from Chlorella dry powder before performing separation and purification; and the CM has a weight-average molecular weight of 1 kDa-16 kDa and a polydispersity index of 1-3.

The present disclosure provides the SMC compound, including an SMC derivative or salt thereof, having a general formula as follows:

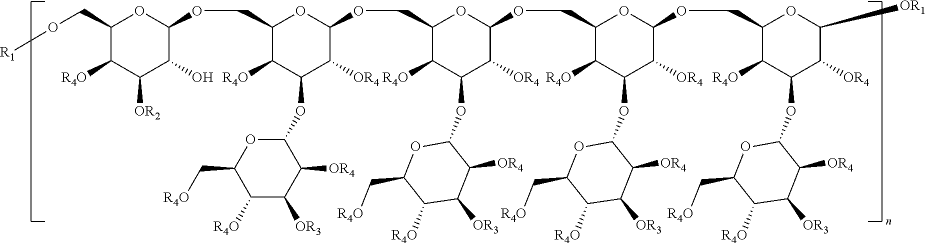

where R₁ is optionally one or more of the H, or —SO₃H or D-β-2,3,4,6-tetrasulfated galactosyl, which are independent of each other; R₂ is optionally one or more of the H, or the —SO₃H, or D-α-2,3,4,6-tetrasulfated mannosyl, or 3-O-methyl-D-α-2,4,6-trisulfated mannosyl, which are independent of each other; R₃ is optionally one or more of the H, or the —CH₃, or the —SO₃H, which are independent of each other; R₄ is optionally one or more of the H or the —SO₃H, which are independent of each other; and n is an arbitrary integer of 1-10.

Preferably, the SCM compound is obtained by sulfation modification of the CM. The SCM compound consists of the mannose, the 3-O-methyl-mannose, and the galactose, where a molar ratio of the mannose to the galactose is (2.0±0.5) to (5.0±1.0). The SCM compound has a weight-average molecular weight of 4 kDa-40 kDa and a polydispersity index of 1-3.5.

Preferably, the SCM compound has a content of sulfate groups of 20%-60%.

The present disclosure provides the SCM compound, which is applied to a pharmaceutical composition for the prevention and/or treatment of thrombotic cardiovascular diseases.

Preferably, the SCM compound is applied to the pharmaceutical composition for prevention and/or treatment of thrombotic cardiovascular diseases, where the thrombotic cardiovascular diseases are formed through venous or arterial thrombosis, or are ischemic cardiovascular and cerebrovascular diseases.

Preferably, the SCM compound is applied to the pharmaceutical composition for prevention and/or treatment of thrombotic cardiovascular diseases, where the pharmaceutical composition is one or more of an SCM derivative or salt thereof, as well as an excipient.

Preferably, the SCM compound is applied to the pharmaceutical composition for prevention and/or treatment of thrombotic cardiovascular diseases, where the salt is one or more of sodium salt, potassium salt, or calcium salt; and the pharmaceutical composition is in a dosage form of a lyophilized powder injection or an aqueous solution for injection.

Therefore, the present disclosure uses the CM or the SCM compound and application of the SCM compound, having the following beneficial effects:
(1) the CM is extracted and separated from Chlorella with strong fecundity, rapid growth rate, and high yield to prepare a polysaccharide sulfate derivative, to replace heparin, is not contaminated by viruses, bacteria, etc., and is low in price.
(2) The structure of the CM is clear and regular, and the structure of SCM prepared by derivatization of the CM is clearer than those of most other reported polysaccharide sulfates, and even the SCM with a uniform structure can be prepared.
(3) For the first time, the CM with a novel and regular structure is extracted, separated, and purified from the Chlorella. The SCM prepared from the CM and pharmaceutically acceptable salt thereof have strong anticoagulant activity, can effectively inhibit FXase of the intrinsic pathway, and can be used for preventing and treating thrombotic cardiovascular disease.

The technical solution of the present disclosure will be further described in detail below through the accompanying drawings and the embodiments.

### BRIEF DESCRIPTION OF THE DRAWINGS

FIG. 1 shows a high-performance liquid chromatogram of CM and SCM in the CM or an SCM compound thereof and application of SCM compound of the present disclosure;

FIG. 2 shows an analytical chromatogram of monosaccharide composition of the CM in the CM or the SCM compound and application of the SCM compound of the present disclosure;

FIG. 3 shows an analytical chromatogram of monosaccharide composition of the SCM in the CM or the SCM compound and application of the SCM compound of the present disclosure;

FIG. 4 shows an infrared spectrum of the CM in the CM or the SCM compound and application of the SCM compound of the present disclosure;

FIG. 5 shows an infrared spectrum of the SCM in the CM or the SCM compound and application of the SCM compound of the present disclosure;

FIG. 6 shows ¹H and ¹³C NMR spectra of the CM in the CM or the SCM compound and application of the SCM compound of the present disclosure;

FIG. 7 shows ¹H and ¹³C NMR spectra of the SCM in the CM or the SCM compound and application of the SCM compound of the present disclosure;

FIG. 8 shows ¹H-¹³C HSQC spectra of the CM in the CM or the SCM compound and application of the SCM compound of the present disclosure;

FIG. 9 shows ¹H-¹³C HSQC spectra of the SCM in the CM or the SCM compound and application of the SCM compound of the present disclosure; and

FIG. 10 shows ¹H-¹H ROESY spectra of the SCM in the CM or the SCM compound and application of the SCM compound of the present disclosure.

### DETAILED DESCRIPTION OF THE EMBODIMENTS

The technical solution of the present disclosure will be further described in detail below through the accompanying drawings and the embodiments.

To make the purpose, technical solution, and advantages of the embodiments of the present disclosure clearer, the technical solutions in embodiments of the present disclosure will be described clearly and completely hereinafter with reference to the accompanying drawings in the embodiments of the present disclosure. Apparently, the embodiments described are merely a part rather than all of the embodiments of the present disclosure. The components of the embodiments of the present disclosure described and shown in the accompanying drawings can be arranged and designed in various configurations.

The present disclosure provides a CM that consists of mannose, 3-O-methyl-mannose, and galactose. A general formula structure of the CM is shown in the following figure:

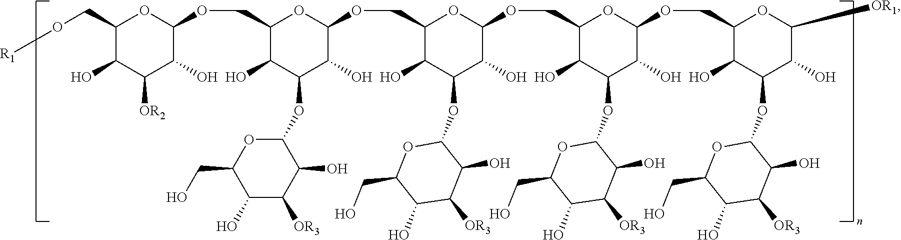

where R₁ is optionally one or more of H or D-β-galactosyl; R₂ is optionally one or more of H, or D-α-mannosyl, or 3-O-methyl-D-α-mannosyl; R₃ is optionally one or more of H or —CH₃; and n is an arbitrary integer of 1-10.

Chlorella of the present disclosure belongs to Chlorella of Chlorellaceae of Chlorocaccales of Chlorophyceae of Chlorophyta, including, but not limited to, C. pyrenoidosa, C. ellipsoidea, C. emersonii, C. kesslerii, and C. vulgaris cultivated in artificial freshwater. It may be understood by those skilled in the art that for the CM meeting a definition of the present disclosure, the CM can also be used for preparing the CM, SCM, or pharmaceutically acceptable salt thereof of the present disclosure, even if it is derived from other green algae species.

The CM is obtained by extracting total polysaccharides from the Chlorella dry powder before performing separation and purification. The weight-average molecular weight of the CM is 1 kDa-16 kDa, and the polydispersity index is 1-3. Further, the weight-average molecular weight of the CM is 1.5 kDa-15 kDa, and a peak molecular weight is preferably 9 kDa-14 kDa.

The present disclosure provides the SMC compound, including an SMC derivative or salt thereof, having a general formula as follows:

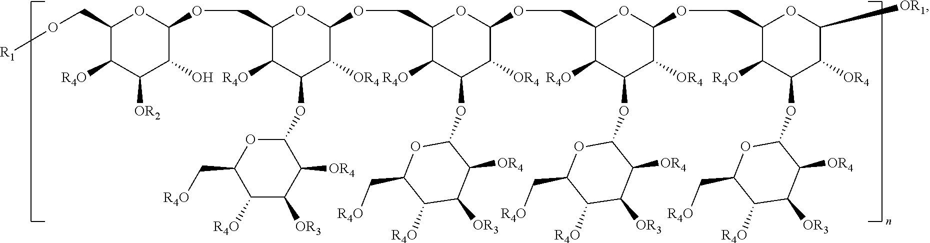

where R₁ is optionally one or more of H, or —SO₃H or D-β-2,3,4,6-tetrasulfated galactosyl, which are independent of each other; R₂ is optionally one or more of the H, or the —SO₃H, or D-α-2,3,4,6-tetrasulfated mannosyl, or 3-O-methyl-D-α-2,4,6-trisulfated mannosyl, which are independent of each other; R₃ is optionally one or more of the H, or —CH₃, or the —SO₃H, which are independent of each other; R₄ is optionally one or more of the H or the —SO₃H, which are independent of each other; and n is an arbitrary integer of 1-10.

The SCM compound is obtained by sulfation modification of the CM. The SCM compound consists of the mannose, the 3-O-methyl-mannose, and the galactose, where a molar ratio of the mannose to the galactose is (2.0±0.5) to (5.0±1.0). The polydispersity index of the SCM compound is 1-3.5. Further, the SCM has a weight-average molecular weight of 4 kDa-40 kDa and a peak molecular weight of preferably 10 kDa-35 kDa. The content of sulfate groups of the SCM compound is 20%-60%. It may be understood by those skilled in the art that in sulfation modification of the CM, sulfation reaction conditions such as reaction time, reaction temperature, and reactant concentration can be controlled to obtain the SCM with any content of the sulfate groups.

According to the present disclosure, extracting and separating the CM from Chlorella can be performed complying with a known method in the art, which generally includes, but is not limited to, the following steps: using a hot water extraction method, or performing ultrasonic/microwave/high pressure assisted extraction, or performing enzymatic hydrolysis-assisted extraction by adding papain; removing small molecular impurities by salting-out alcohol precipitation, dialysis, ultrafiltration or gel filtration, and removing proteins by Sevag or isoelectric precipitation; purifying a polysaccharide extract, where the purifying step optionally uses ion exchange chromatography, gel column chromatography, etc.; and finally, performing drying to obtain purified CM.

The SCM of the present disclosure is prepared from the CM by using a sulfonation reaction and introducing the sulfate group. A method used in the sulfonation reaction can be, but not limited to, a sulfur trioxide-pyridine method, a sulfur trioxide-trimethylamine method, etc., that is, the CM is used as a receptor, and sulfur trioxide is used as a sulfate donor. The purifying step of the SCM after the sulfonation reaction optionally uses anion exchange column chromatography, quaternary ammonium salt precipitation, and ultrafiltration to obtain purified SCM.

In the present disclosure, the pharmaceutically acceptable salt can be in a pharmaceutically acceptable single salt form (such as sodium salt, potassium salt, or calcium salt) obtained by conversion from the SCM by a cation exchange column. In a salt formation process, the cation exchange column can be pretreated for an exchange into the corresponding salt, and then a sample is loaded for an exchange to form the salt. The SCM can also be exchanged into a hydrogen form by the cation exchange column, and then the corresponding alkali is used for neutralization to obtain salt corresponding to the SCM. The step can be performed complying with a conventional method.

In the present disclosure, characteristic absorption peaks of the CM in a Fourier transform infrared spectrum are: 3404±50 cm⁻¹, 2933±50 cm⁻¹, 1647±50 cm⁻¹, 1379±50 cm⁻¹, 1258±50 cm⁻¹, 1054±50 cm⁻¹ and 548±50 cm⁻¹. Compared with that of the CM, an infrared spectrum of the SCM is significantly enhanced at the characteristic absorption peaks of 1259±50 cm⁻¹, 821±50 cm⁻¹, etc.

In the present disclosure, a ¹H NMR (600 MHZ, D₂O, 298 K) spectrum of the CM contains following signal peaks: 5.11±0.3 ppm, 5.07±0.3 ppm, 4.79±0.3 ppm, 4.55±0.3 ppm, 4.51±0.3 ppm, 4.27±0.3 ppm, 4.09±0.3 ppm, 4.02±0.3 ppm, 3.98±0.3 ppm, 3.94±0.3 ppm, 3.93±0.3 ppm, 3.92±0.3 ppm, 3.91±0.3 ppm, 3.89±0.3 ppm, 3.82±0.3 ppm, 3.81±0.3 ppm, 3.79±0.3 ppm, 3.76±0.3 ppm, 3.74±0.3 ppm, 3.73±0.3 ppm, 3.71±0.3 ppm, 3.70±0.3 ppm, 3.65±0.3 ppm, 3.56±0.3 ppm, 3.48±0.3 ppm, 3.42±0.3 ppm, and 2.17±0.3 ppm. Most of the ¹H NMR signal peaks of the SCM are shifted downfield obviously.

In the present disclosure, a ¹³C NMR (600 MHZ, D₂O, 298 K) spectrum of the CM contains following signal peaks: 103.4±0.5 ppm, 103.1±0.5 ppm, 96.1±0.5 ppm, 79.7±0.5 ppm, 76.1±0.5 ppm, 73.5±0.5 ppm, 72.7±0.5 ppm, 70.7±0.5 ppm, 70.3±0.5 ppm, 70.2±0.5 ppm, 69.3±0.5 ppm, 68.6±0.5 ppm, 66.6±0.5 ppm, 65.9±0.5 ppm, 65.6±0.5 ppm, 64.2±0.5 ppm, 60.9±0.5 ppm, and 56.3±0.5 ppm. Most of the ¹³C NMR signal peaks of the SCM are shifted downfield obviously.

The SCM compound of the present disclosure is applied to the prevention and/or treatment of thrombotic cardiovascular disease, such as thrombotic cardiovascular and cerebrovascular diseases, deep vein thrombosis, pulmonary venous thrombosis, peripheral venous thrombosis, and peripheral arterial thrombosis. The SCM compound can be used as a pharmaceutical composition which is one or more of an SCM derivative or salt thereof, and an excipient. The salt is one or more of sodium salt, potassium salt, or calcium salt. The pharmaceutical composition is in a dosage form of a lyophilized powder injection or an aqueous solution for injection.

### Example 1. Preparation of CM

### (1) Experimental Materials

Commercially available Chlorella pyrenoidosa dry powder; DEAE Sepharose Fast Flow, Sepharose CL-6B, Sephadex G-25 and other products from GE Healthcare, America; α-amylase and other products from Sigma, America; and other reagents such as sodium chloride, ethanol, trichloromethane, an iodine solution, and n-butanol, which were commercially available analytically pure reagents.

### (2) Extraction Method

30 g of Chlorella dry powder was added to 600 mL of pure water for uniform mixing; a mixture was extracted in a water bath at 90° C. for 3 h; extraction was repeated twice; supernatants obtained by centrifugation at 4700 rpm for 15 min were combined; 200 mg of the α-amylase was added for stirring in a water bath at 50° C.; and a reaction was performed until a reaction solution was not changed into blue through detection with the iodine solution. Boiling was performed for enzyme denaturalization for 15 min; centrifugation was performed at 4500 rpm for 20 min; anhydrous ethanol was added to the supernatants until a final concentration of the ethanol was 75%; 10 mL of a saturated sodium chloride solution was added dropwise; and a mixed solution was placed in a refrigerator at 4° C. for standing for 12 h. Centrifugation was performed at 4700 rpm for 15 min; precipitates were redissolved with pure water; a Sevag reagent prepared by mixing the trichloromethane with the n-butanol at a volume ratio of 5:1 was added; a sample solution was mixed with the Sevag reagent at a volume ratio of 5:1 for shaking for 20 min; then a centrifugation was performed at 4500 rpm for 20 min; a supernatant aqueous solution was sucked; an organic layer and a denatured protein solid were discarded; and the operation was repeated until the proteins were completely removed. The supernatants were concentrated and lyophilized to obtain the total polysaccharides from the Chlorella.

### (3) Separation and Purification

Total polysaccharide lyophilized powder was taken, and distilled water was added for dissolving the powder; and separation and purification were performed by a DEAE Sepharose Fast Flow anion exchange column (40 cm×3 cm) at an elution flow rate of 2 mL/min, to obtain a water elution fraction of Chlorella polysaccharides. Sephadex G-25 was used for desalination and lyophilization. A lyophilized sample was dissolved into 0.2 M sodium chloride solution; separation and purification were performed by a Sepharose CL-6B (150 cm×1.5 cm), and eluting was performed with a 0.2 M NaCl solution at an elution rate of 0.33 mL/min; a sample was collected in per tubes of 5 mL; and the collected samples were desalted by Sephadex G-25 to obtain the purified CM.

### (4) Experimental Results

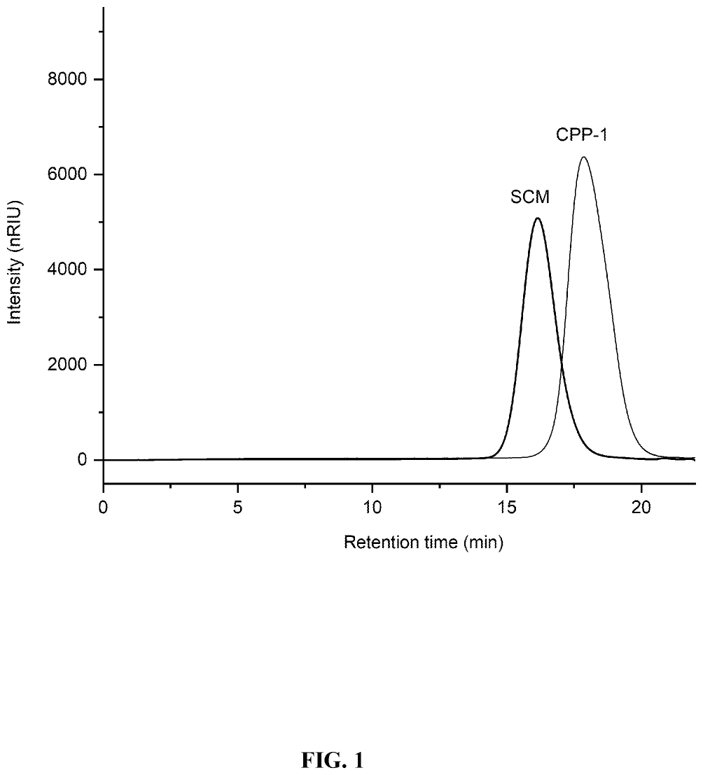

The yield of the total polysaccharides from Chlorella was about 1.64% based on the Chlorella dry powder, and the total polysaccharides from Chlorella were purified by column chromatography to obtain the CM. As shown in FIG. 1, a high-performance liquid chromatography (HPLC) peak of the CM was a single symmetrical peak, suggesting that the purity of the separated and purified CM was high, and calculated by an area normalization method to be larger than 99%.

### Example 2 Preparation of SCM

### (1) Experimental Materials and Reagents

CM was prepared in Example 1; and anhydrous DMSO, sulfur trioxide-pyridine, ethanol, sodium chloride, etc. were all commercially available analytical pure reagents.

### (2) Sulfation Modification Method

100 mg of the CM was added to 10 mL of the anhydrous DMSO for magnetic stirring until complete dissolution; 1.324 g of sulfur trioxide-pyridine was added for stirring reaction at 50° C. for 30 min or 60 min; after completion of the reaction, the anhydrous ethanol and 1 mL of saturated sodium chloride were added; a mixture was placed in a refrigerator at 4° C. for standing for more than 4 h, and then centrifuged at 4700 rpm; and salt was removed through repeated salting-out alcohol precipitation.

The sulfated sample was dissolved into deionized water; a resultant was filtered by a 0.45 μm microporous membrane, purified by a DEAE Sepharose Fast Flow anion exchange chromatographic column (40 cm×3 cm), eluted with pure water, a 0.3 M NaCl solution, and a 2.0 M NaCl solution in turn, and detected by a phenol-sulfuric acid method; and a 2.0 M eluent was collected, and desalted by dialysis with a dialysis bag with a molecular weight cut-off of 3.5 KDa for 72 h, and concentration and lyophilization were performed, to finally prepare the SCM with different contents of the sulfate groups. The cation exchange column (20 cm×2 cm) was converted to a hydrogen form with 1 M hydrochloric acid; and then the prepared SCM aqueous solution was exchanged into the hydrogen form through the column, and then neutralization was performed with sodium hydroxide, potassium hydroxide or calcium hydroxide to obtain the sodium salt, the potassium salt or the calcium salt corresponding to the SCM.

### (3) Results

Yields of the SCM-1 and the SCM-2 prepared by the sulfonation reaction for 30 min and the sulfonation reaction for 60 min were 120% and 148% based on the CM as the raw material respectively. As shown in FIG. 1, prepared SCM-1 was analyzed as a single chromatographic peak with HPLC, and the retention time of the SCM-1 was shorter than that of the CM, suggesting that the molecular weight of the SCM became larger.

### Example 3 Physicochemical Properties and Composition Analysis on CM and SCM

### (1) Experimental Materials and Reagents

Glucuronic acid (GlcA), L-rhamnose monohydrate (Rha), D-galacturonic acid (GalA), galactose (Gal), glucose (Glc), 1-phenyl-3-methyl-5-pyrazolone and other products from Sigma, America; a pullulan standard substance purchased from Shanghai Aladdin Biochemical Technology Co., Ltd.; other reagents such as mannose (Man), ribose (Rib), arabinose (Ala), xylose (Xyl), fucose (Fuc), acetonitrile, barium chloride, Coomassie brilliant blue, trifluoroacetic acid, and potassium iodide, which were commercially available analytically pure reagents.

### (2) Experimental Method

The contents of sulfate groups in the CM and the SCM were determined by a classical turbidimetric method (Dodgson and Price, Biochem. J. 1962, 84, 106-110). Their contents of the proteins were determined by the Coomassie brilliant blue (Bradford. Anal. Biochem. 1976, 72, 248-254).

Molecular weights were determined by high-performance gel permeation chromatography (HPGPC) using a LC-2030C 3D Plus HPLC system (Shimadzu Corporation, Kyoto, Japan). A detector used was a differential refractive index detector; and a chromatographic column: a Shodex OHpak SB-804 HQ column (7 μm, 8×300 mm). Chromatographic conditions: flow rate was 0.5 mL/min, a mobile phase was a 0.1 M NaCl aqueous solution, and column temperature was 35° C. Molecular weights of the pullulan standard substance were 344.0 kDa, 107.0 kDa, 47.1 kDa, 21.1 kDa, and 9.6 kDa, respectively. A standard curve was drawn, and the molecular weight of a sample was calculated.

The monosaccharide composition was analyzed by a pre-column derivatization HPLC method, where the detector used was a DAD detector; a chromatographic column used was an Agilent ZORBAX Eclipse Plus C18 column (4.6×250 mm, 5 μm); a detection wavelength was 245 nm; the column temperature was 30° C.; the mobile phase was acetonitrile and a 0.1 M PBS solution (17:83, v/v); pH value was 6.7; the flow rate was 1.0 mL/min; and an injection volume was 20 μL. Before analysis on standard monosaccharide and polysaccharide samples, 4.0 M trifluoroacetic acid was used for hydrolyzation at 120° C. for 2 h, and an obtained hydrolyzed sample was subjected to PMP derivatization, and then analyzed by HPLC. Linear regression and fitting were used to calculate the molar percentages of various monosaccharides.

The glycosidic linkage types of the CM were analyzed by a methylation-GC-MS method. 5 mg of the polysaccharide samples were taken and put in a round-bottom flask, and 2 mL of anhydrous DMSO was added for dissolving the polysaccharide samples; and then about 50 mg of NaOH was added for full dissolution; then treatment was performed in an ice bath; 1.5 mL of CH₃I was added for a reaction for 2 h; 2 mL of pure water was added to terminate the reaction; extraction was performed, and then drying was performed; and the above operations were repeated to enable the polysaccharide to be fully methylated. 3 mL of a 4 M TFA solution was added to a methylated sample for hydrolyzation at 120° C. for 2 h; reduced pressure evaporation was performed to dryness; 3 mL of methanol was added; then reduced pressure evaporation was performed to dryness; and the above operations were repeated for 3 times. The sample was dissolved with 2 ml of the pure water, and pH was adjusted to be alkaline; 30 mg of NaBH₄ was added for reduction at room temperature for 3 h; and glacial acetic acid was added dropwise until the pH of the solution was acidic so as to remove excess NaBH₄. After the end of the reaction, the methanol was repeatedly added for concentration to dryness under the reduced pressure; further drying was performed in a vacuum drying oven for 24 h; 1.5 mL of acetic anhydride and 1.5 mL of pyridine were added for a reaction at 100° C. for 1 h; 1.5 mL of water was added to a reaction solution; then 3 mL of dichloromethane was added for extraction; and an extract was washed with 2 mL of pure water for 3 times, subjected to rotary evaporation to dryness, and dissolved with 100 μL of the dichloromethane for GC-MS analysis. A capillary chromatographic column was SP-2330 ms (30 m×0.25 mm×0.2 μm, Supelco, America); carrier gas used was He; a heating program: 160-210° C. (2/min° C.), 210-240° C. (5/min° C.); and an ionization mode was EI (70 kV).

The polysaccharides were subjected to infrared spectroscopy by Nicolet iS50 Fourier transform infrared spectrometer (Thermo Fisher Scientific, America). The sample and KBr particles were ground, mixed, and pelleted with a scanning range of 4000-400 cm⁻¹, to obtain an infrared spectrogram.

1D and 2D nuclear magnetic resonance spectra of the polysaccharides were determined by a Bruker Avance 600 MHz nuclear magnetic resonance spectrometer. The dried polysaccharide samples were dissolved in deuterium oxide (D₂O, 99.9% D) for vacuum drying; and after repeated D₂O exchange, D₂O containing an internal standard was added. The concentration of the samples was 10-20 mg/mL for determining the 1D and 2D nuclear magnetic resonance spectra, such as 1H, 13C, COSY, TOCSY, ROESY, HSQC, and HMBC.

### (3) Experimental Results

The weight-average molecular weight of the CM was 13.6 kDa, and no sulfate group and protein were detected. The contents of sulfate groups in the samples SCM-1 and SCM-2 (reacted for 30 min and 60 min, respectively) were 40.2%±0.1% and 54.5%±0.7% respectively; and the samples SCM-1 and SCM-2 did not contain proteins.

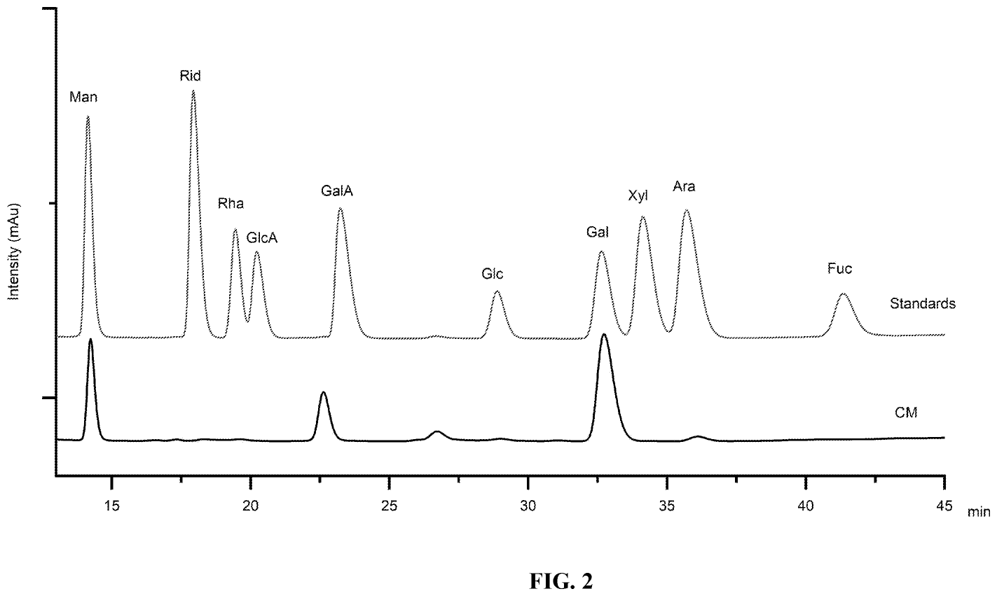

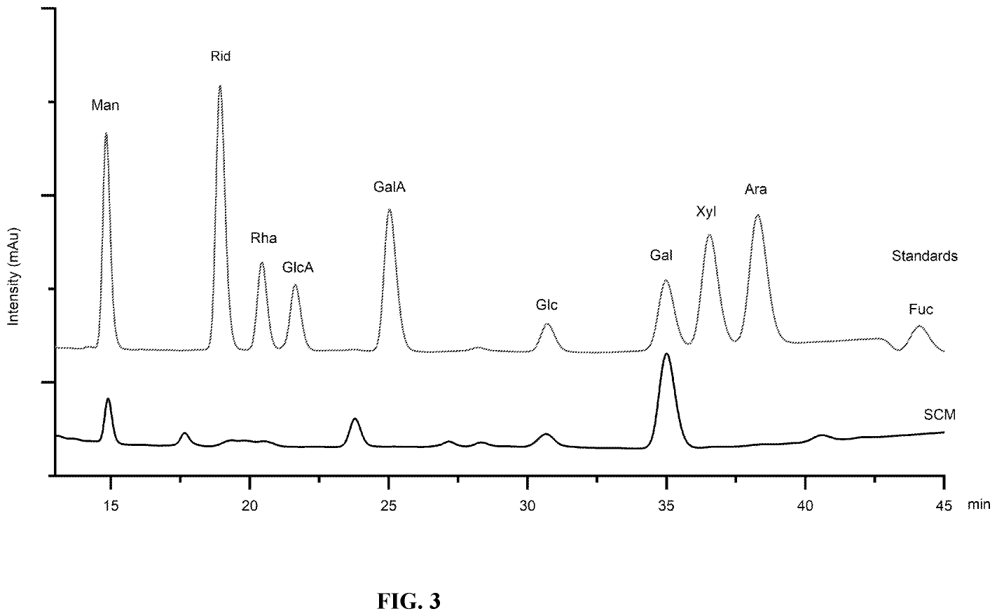

As shown in FIG. 2, the chromatogram of the CM mainly consisted of a mannose chromatographic peak, a galactose chromatographic peak, and an unknown peak. The molar ratio of the mannose to the galactose was 4.4:10.5. The unknown peak was determined as 3-O-methyl-mannose by high-resolution mass spectrometry. As shown in FIG. 3, the monosaccharide composition of the SCM-1 was consistent with that of the CM, and there was no significant change.

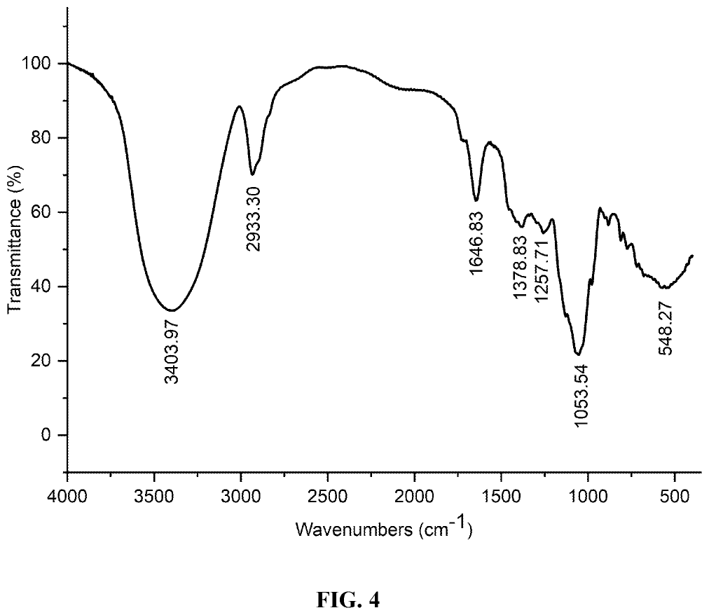

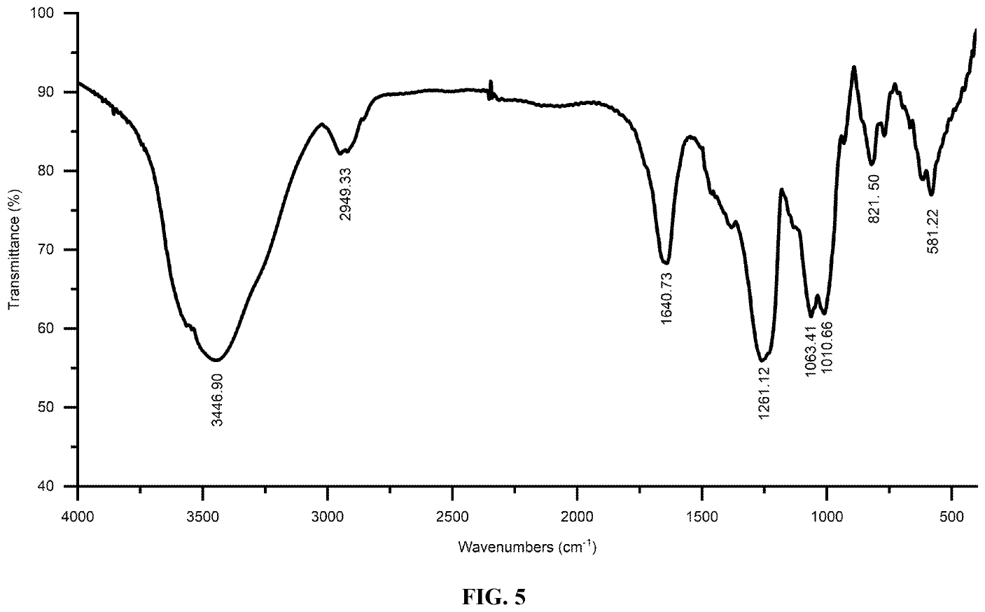

The infrared spectra of the CM and the SCM-1 are shown in FIGS. 4-5. A signal peak at 3403.97 cm⁻¹ was attributed to stretching vibration of —OH; a characteristic peak at 2933.30 cm⁻¹ was attributed to stretching vibration of a C—H bond; 1646.83 cm⁻¹ was a signal of water; and a characteristic peak near 1053.54 cm⁻¹ was formed due to stretching vibrations of C—C and C—O. The new appeared strong absorption peaks at 1261.12 cm⁻¹ and 821.50 cm⁻¹ were in the infrared spectrum of the SCM compared with the CM, being formed by stretching vibration of S═O and bending vibration of C—O—S in the sulfate groups.

The results of CM methylation are shown in Table 1. The polysaccharides contained three glycosidic linkage types, including Manp(1→, →6)Galp(1→, and →3,6)Galp(1→), with a molar ratio of 3.9:1.0:3.5.

TABLE 1
Results of Detection with Methylation-GC-MS

| Methylated monosaccharide | Glycosidic linkage | Molar | Relative |
|---|---|---|---|
| derivatives detected by GC-MS | types | ratio | retention time |
| 2,3,4,6-tetra-O-Me-Manp | Manp(1→ | 3.9 | 1.0 |
| 2,3,4-tri-O-Me-Galp | →6)Galp(1→ | 1.0 | 1.7 |
| 2,3-di-O-Me-Galp | →3,6)Galp(1→ | 3.5 | 2.1 |

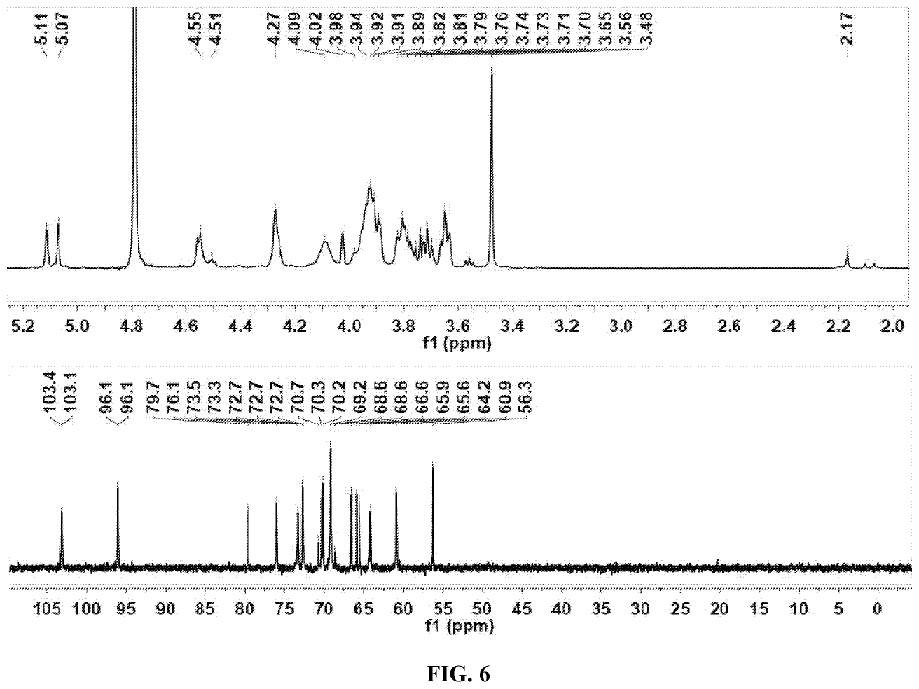

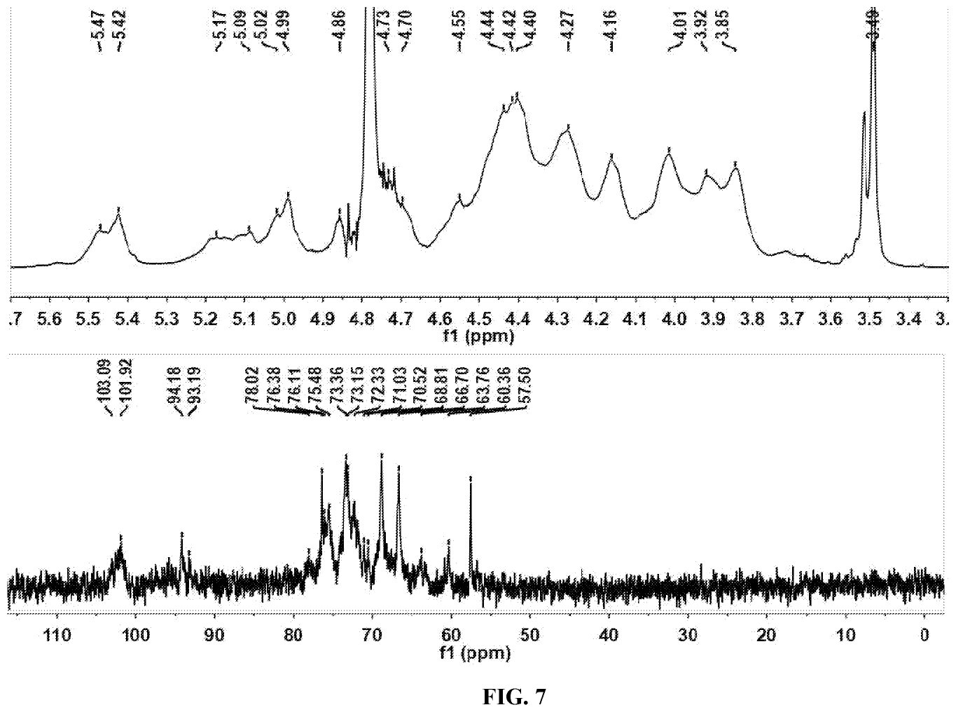

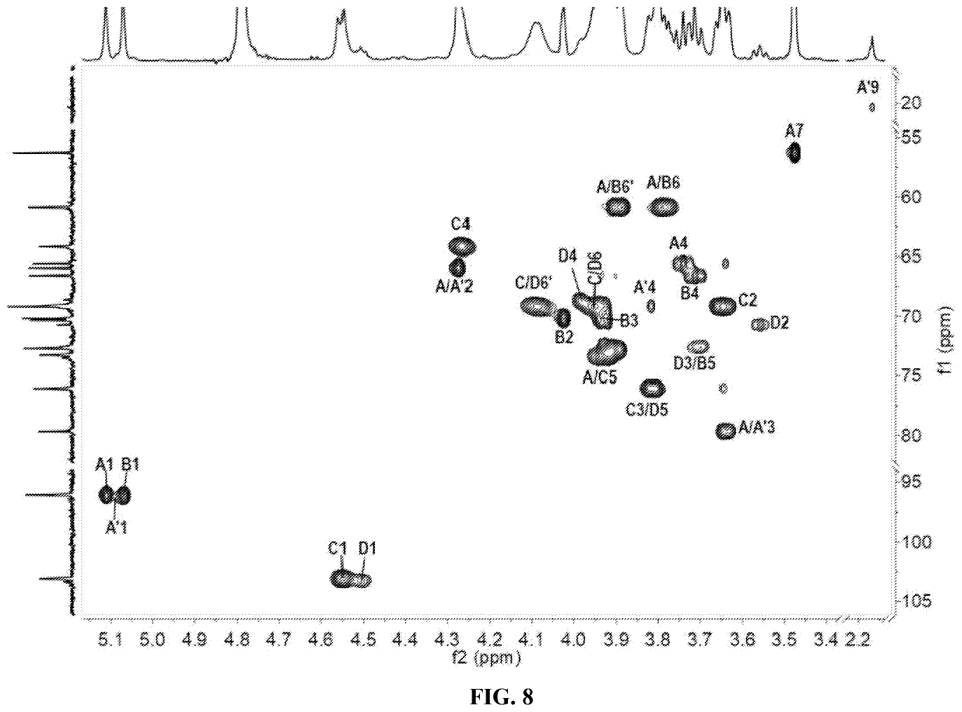

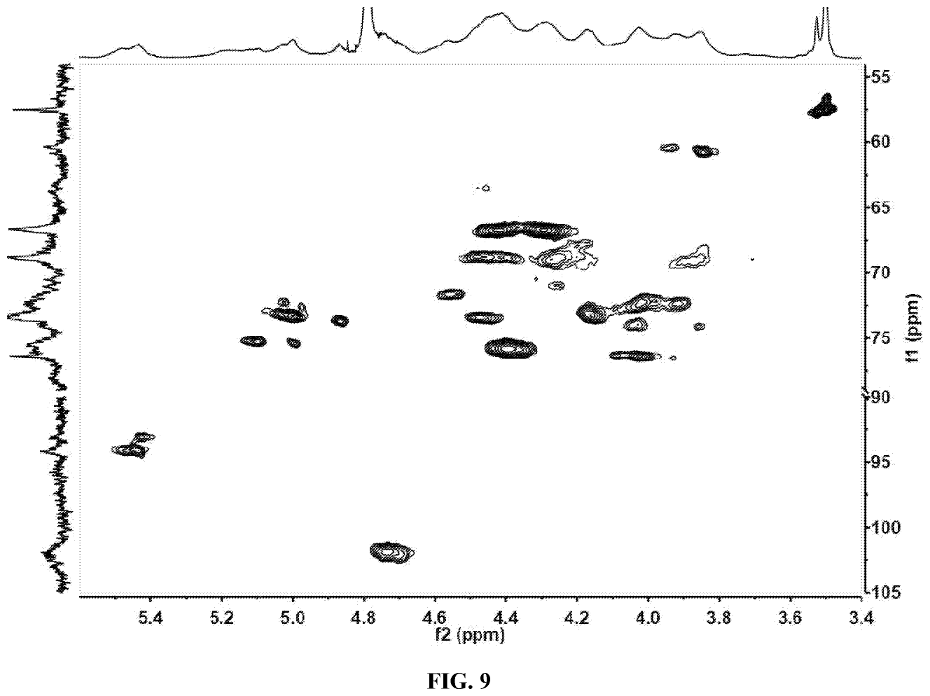

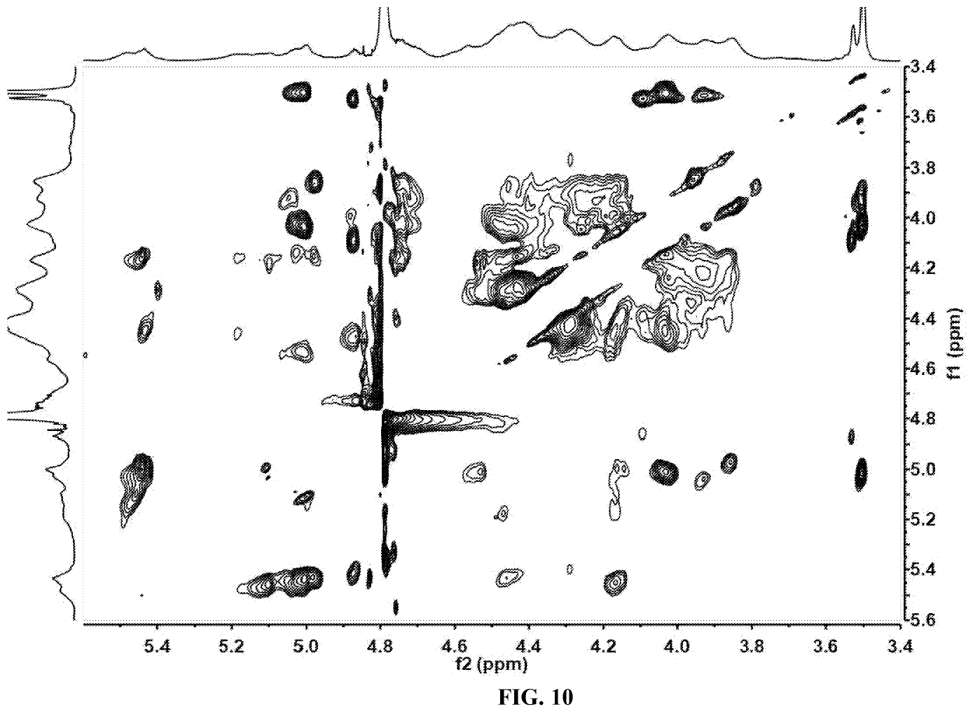

¹H, ¹³C, and HSQC nuclear magnetic resonance spectra of the CM and the SCM-1 are shown in FIGS. 6-10. Attribution results of chemical shift signals in the ¹H and ¹³C NMR of the CM are shown in Table 2. Based on ¹H, ¹³C, and two-dimensional correlation spectra, the structures of the CM and the SCM can be analyzed. Chemical structural formulas of the CM and the SCM have structural characteristics described in the formulas (I) and (II) of the present disclosure. A main chain of each of the CM and the SCM consists of (1→6)-linked β-D-Galp, which is substituted by D-Man and 3-O-methyl-D-Man (with a molar ratio of 1:1) on C-3. There is a small quantity of acetyl substitutions at the 6 position of the 3-O-methyl-D-Man (with a molar percentage less than 5%). According to a proton signal integral of ¹H NMR, the level of acetylation could be calculated as approximately 2.1%. The CM and SCM are mannogalactan and sulfated mannogalactan with a novel and regular structure, respectively. The sulfate groups of the SCM can be substituted at any hydroxyl site of the CM.

TABLE 2
Attribution Results of Chemical Shift Signals in ¹H and ¹³C NMR of CM

| | | Chemical Shifts (δ, ppm) | | | | | | | | |
|---|---|---|---|---|---|---|---|---|---|---|
| | | | | | | | | 7 | | 9 |
| Residues | H/C | 1 | 2 | 3 | 4 | 5 | 6 | (3-O-Me) | 8 | (6-O-Ac) |
| (t-3-O-Me-α-Manp, A) | H | 5.11 | 4.27 | 3.64 | 3.75 | 3.92 | 3.79/ | 3.47 | | |
| | | | | | | | 3.90 | | | |
| | C | 96.1 | 65.9 | 79.7 | 65.6 | 72.7 | 60.9 | 56.4 | | |
| (t-6-O-Ac-3-O-Me-α- | H | 5.09 | 4.28 | 3.65 | 3.82 | 4.14 | 4.42/ | 3.42 | | 2.17 |
| Manp, A′) | | | | | | | 4.33 | | | |
| | C | 96.3 | 65.9 | 79.6 | 69.2 | 70.5 | 63.4 | 56.7 | 177.8 | 24.5 |
| (t-α-Manp, B) | H | 5.07 | 4.02 | 3.93 | 3.69 | 3.72 | 3.78/ | | | |
| | | | | | | | 3.89 | | | |
| | C | 96.1 | 70.2 | 70.3 | 66.6 | 72.7 | 60.9 | | | |
| (1,3,6-β-Galp, C) | H | 4.56 | 3.65 | 3.81 | 4.26 | 3.92 | 3.94/ | | | |
| | | | | | | | 4.07 | | | |
| | C | 103.1 | 69.2 | 76.1 | 64.2 | 73.3 | 69.1 | | | |
| (1,6-β-Galp, D) | H | 4.51 | 3.56 | 3.70 | 3.98 | 3.81 | 3.93/ | | | |
| | | | | | | | 4.10 | | | |
| | C | 103.3 | 70.7 | 72.6 | 68.7 | 76.1 | 69.3 | | | |

### Example 4 Determination of Anticoagulant Activity of SCM

### (1) Experimental Materials and Reagents

The SCM sample used was SCM-1 prepared by a sulfonation reaction for 30 min in Example 2; enoxaparin sodium (LMWH, Mw was about 4500 Da) from Sanofi, France; coagulation control plasma from TECOGmbH, German; Tris-HCl (with purity greater than 99.5%) from Amresco; assay kits for activated partial thromboplastin time (APTT), prothrombin time (PT), thrombin time (TT), etc., from TECO GmbH, German; calcium chloride from TECO GmbH, German; an FVIII: C kit from HYPHEN BioMed, France; and a Factor VIII from Bayer, German.

### (2) Experimental Instruments

MC-4000 coagulometer (TICO GmbH, German); and Victor Nivo Multimode Plate Reader (PerkinElmer, America).

### Experimental Method

(3) Determination of APTT, TT, and PT: influences of the sample on the PT, the TT, and the APTT of standard human plasma were detected complying with an instruction of the kit. 5 μL of the SCM with a series of concentrations was accurately sucked by a pipette, and added to test tubes preheated to 37° C.; then 45 μL of the coagulation control plasma was added for incubation at 37° C. for 2 min; and 50 μL of a PT, TT or APTT reagent after slight shaking was added to the test tubes. During the determination of the PT or the TT, time counting was performed immediately, and coagulation time was recorded. During the determination of the APTT, incubation was needed at 37° C. for 3 min, and then 50 μL of a 0.02 mol/L calcium chloride solution preheated to 37° C. was added; time counting was performed; and coagulation time was recorded. A Tris-HCl buffer was used as a control according to the above operation.

### (4) Determination of Anti-FXase Activity

Detection was performed complying with a method in an instruction of the FVIII: C kit. 30 μL of the SCM samples with gradient concentrations were added to a 96-well plate, 30 μL of a 2 IU/mL VIII solution and 30 μL of an R₂ solution (a 60 nM factor IXa solution, containing Ca²⁺, IIa and PC/PS) were added, shaking and uniform mixing were performed by a microplate reader; incubation was performed at 37° C. for 2 min; 30 μL of an R₁ solution (a 50 nM factor X solution, containing a factor IIa inhibitor) was added, shaking and uniform mixing were performed by the microplate reader, and incubation was performed at 37° C. for 1 min; finally, 30 μL of an R₃ solution (containing a 8.4 mmol/L factor Xa chromogenic substrate SXa-11) preheated to 37° C. was added; a mixture was placed in the microplate reader for shaking and uniform mixing; an OD₄₀₅ value was read every 31 s; and continuous detection was performed for 5 min. A Tris-HCl buffer was used as a negative control complying with the above operation, and a change rate of the OD₄₀₅ value was used to express the production quantity of a factor Xa and the activity of a FXase.

### (5) Results

The SCM had strong anticoagulant activity, and prolonged the APTT, the TT, and the PT in a dose-dependent manner. Particularly, a drug concentration required to double the APTT was lower than that of the enoxaparin sodium as the low molecular weight heparin (LMWH). The SCM had significant inhibitory activity against the FXase, and an EC50 value reaches 13.65 ng/mL, which was stronger than that of the enoxaparin sodium (Table 3).

TABLE 3
Anticoagulant Activities of CM and SCM

| | APTT ^{a} | TT ^{a} | PT ^{a} | Anti-Xase |
|---|---|---|---|---|
| Sample | (μg/mL) | (μg/mL) | (μg/mL) | (ng/mL) |
| CM | >128 | >128 | >128 | >2000 |
| SCM | 2.75 ± 0.02 | 7.92 ± 0.29 | 16.83 ± 1.11 | 13.65 ± 1.72 |
| Heparin | 0.81 ± 0.02 | 0.52 ± 0.01 | 3.27 ± 0.06 | 6.49 ± 1.54 |
| Enoxaparin sodium | 3.01 ± 0.04 | 2.44 ± 0.06 | >128 | 37.02 ± 5.17 |

Note: a represents a drug concentration required to double the APTT, the TT, or the PT.

### Example 5 Preparation of SCM Lyophilized Powder Injection

### (1) Experimental Materials and Reagents

SCM used was a sample prepared by sulfonation reaction for 15 min in Example 2; and water for injection.

### (2) Experimental Instruments

Telstar LyoQuest-55 vacuum freeze-dryer

### (3) Experimental Method

5 g of the sodium salt of the SCM was weighed, and 100 mL of the water for injection was added to dissolve the sodium salt; suction filtration was performed with a 22 μm microporous membrane to remove a heat source; a resultant was separately charged in 2 mL vials with 0.5 mL per vial; the vials were half plugged, and placed in a vacuum freeze-dryer with the temperature set at −40° C. and maintained for 4 h, then vacuumizing was performed to 30 Pa to be maintained for 12 h, and then heating was performed to 20° C.; a vacuum degree was reduced to the lowest value that an instrument could achieve, and the lowest value was maintained for 24 h; and the vials were lyophilized, plugged, and capped.

Therefore, the SCM of the present disclosure has strong anticoagulant activity. An IC₅₀ value required to double the APTT of the human control plasma is 2-3 ng/ml, which is equivalent to the Enoxaparin sodium. An EC₅₀ value of inhibiting an activity of an endogenous factor Xase (FXase) is 10-20 ng/mL. The SCM can strongly inhibit the intrinsic pathway, and inhibition in the intrinsic pathway does not affect a physiological hemostasis function and has a lower bleeding tendency.

The SCM and pharmaceutically acceptable salt thereof of the present disclosure have strong anticoagulant activities, thus being applied to the prevention and treatment of thrombotic cardiovascular diseases, such as thrombotic cardiovascular and cerebrovascular diseases, deep vein thrombosis, pulmonary venous thrombosis, peripheral venous thrombosis, and peripheral arterial thrombosis.

The SCM and the pharmaceutically acceptable salt thereof of the present disclosure have good water solubility. Therefore, the present disclosure provides an effective anticoagulant dose of the SCM and the pharmaceutically acceptable salt thereof, as well as a drug excipient for a pharmaceutical composition. The pharmaceutical composition may be in a dosage form of a lyophilized powder injection or an aqueous solution for injection.

Finally, it should be noted that the above embodiments are merely provided for describing the technical solutions of the present disclosure, but not intended to limit the present disclosure. It should be understood by persons of ordinary skill in the art that although the present disclosure has been described in detail with reference to the embodiments, modifications or equivalent replacements can be made to the technical solutions described in the present disclosure, without departing from the spirit and scope of the present disclosure.

What is claimed is:

1. Chlorella mannogalactan (CM), consisting of mannose, 3-O-methyl-mannose, and galactose, having a general formula as follows: 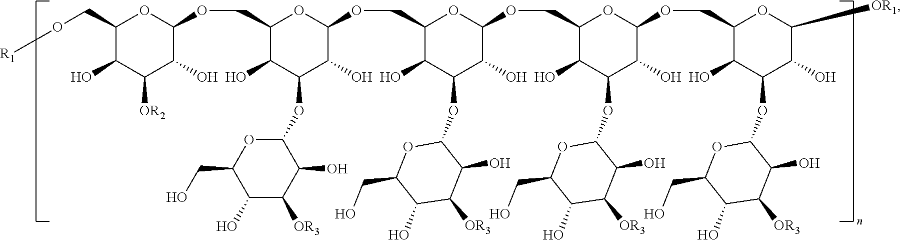
wherein R₁ is one or more of H or D-β-galactosyl;
R₂ is one or more of the H or D-α-mannosyl or 3-O-methyl-D-α-mannosyl;
R₃ is one or more of the H or —CH₃;
n is an integer of 1-10; and
the CM has a peak molecular weight of 9 kDa-14 kDa and a polydispersity index of 1-3.

2. The CM according to claim 1, wherein raw materials for preparing the CM are one or more of Chlorella pyrenoidosa dry powder, Chlorella ellipsoidea dry powder, Chlorella emersonii dry powder, Chlorella kesslerii dry powder, and Chlorella vulgaris dry powder; and
the CM is obtained by extracting total polysaccharides from Chlorella dry powder before performing separation and purification.

3. A sulfated Chlorella mannogalactan (SCM) compound, comprising an SCM derivative or pharmaceutically acceptable salt thereof, having a general formula as follows: 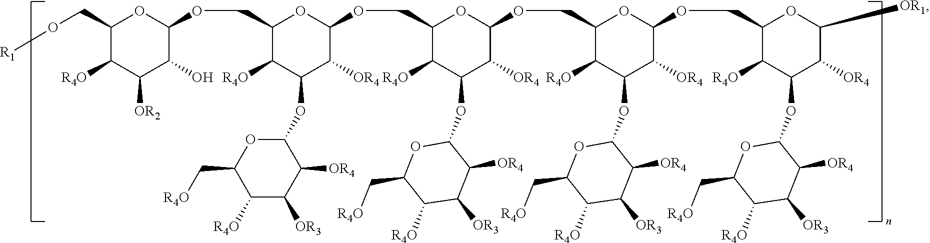
wherein R₁ is one or more of H or —SO₃H or D-β-2,3,4,6-tetrasulfated galactosyl, which are independent of each other;
R₂ is one or more of the H or the —SO₃H or D-α-2,3,4,6-tetrasulfated mannosyl or 3-O-methyl-D-α-2,4,6-trisulfated mannosyl;
R₃ is one or more of the H or —CH₃ or the —SO₃H, which are independent of each other;
R₄ is one or more of the H or the —SO₃H;
n is an integer of 1-10;
the SCM has a peak molecular weight of 10 kDa-35 kDa and a polydispersity index of 1-3, and the SCM compound has a content of sulfate groups of 20%-60%;
the SCM compound is obtained by sulfation modification of the CM; and
the SCM compound consists of mannose, 3-O-methyl-mannose, and galactose, wherein a molar ratio of the mannose to the galactose is (2.0±0.5) to (5.0±1.0).
